(19) United States
(12) Patent Application Publication
(10) Pub. No.: US 2025/0108072 A1
(43) Pub. Date: Apr. 3, 2025

(54) NON-NATURALLY OCCURING THREE-DIMENSIONAL (3D) BROWN ADIPOSE-DERIVED STEM CELL AGGREGATES, AND METHODS OF GENERATING AND USING THE SAME

(71) Applicant: BioRestorative Therapies, Inc., Melville, NY (US)

(72) Inventor: Francisco Javier Silva, Melville, NY (US)

(21) Appl. No.: 18/829,438

(22) Filed: Sep. 10, 2024

Related U.S. Application Data
(63) Continuation of application No. 16/862,226, filed on Apr. 29, 2020, now abandoned.
(60) Provisional application No. 62/840,096, filed on Apr. 29, 2019.

(51) Int. Cl. A61K 35/35; A61K 47/42; A61P 3/00; A61P 3/10; C12N 5/077; C12N 5/0775

(52) U.S. Cl. CPC A61K 35/35; A61K 47/42; A61P 3/00; A61P 3/10; C12N 5/0653; C12N 5/0667; C12N 2501/39; C12N 2501/395; C12N 2501/73; C12N 2501/999; C12N 2513/00

(57) ABSTRACT

The present application provides non-naturally occurring 3D brown adipose-derived stem cell (BADSC) aggregates, methods of making the 3D BADSC aggregates, and methods of using the 3D BADSC aggregates.

### RELATED APPLICATIONS

This application claims the benefit of U.S. Provisional Application No. 62/840,096 filed Apr. 29, 2019, which is incorporated herein in its entirety.

### FIELD

The present application provides non-naturally occurring 3D brown adipose-derived stem cell (BADSC) aggregates, methods of making the 3D BADSC aggregates, and methods of using the 3D BADSC aggregates.

### BACKGROUND

The prevalence of metabolic disorders, such as obesity, has increased dramatically over the past several decades and has become a pandemic. By 2030, more than 50% of Americans will suffer from obesity, resulting in over a 500 billion dollar loss in economic productivity. Obesity is a major risk factor for type II diabetes mellitus, hypertension, cardiovascular disease, osteoarthritis, and certain forms of cancer. Current therapeutic approaches, such as caloric restriction and exercise, which rely mainly on patient's will power to reduce energy intake and/or increase energy expenditure, are of limited effectiveness in obese patients. Bariatric surgery is the only clinically proven therapy in terms of weight loss and decreased morbidity/mortality in patients with a body mass index (BMI) over 40; however, it has associated risks, high costs, and requires proper management of patient's nutrition and physical activity. Despite efforts from researchers and medical professionals worldwide who have been trying to address obesity and other metabolic disorders, there is still a need for alternative ways to increase energy expenditure that could augment the current therapeutic options for treating obese patients and patients with other metabolic disorders.

### SUMMARY

This section provides a general summary of the disclosure, and is not comprehensive of its full scope or all of its features.

Provided herein is a non-naturally occurring three-dimensional brown adipose derived stem cell aggregate. The three-dimensional brown adipose derived stem cell aggregate comprises brown adipose-derived stem cells that express one or more brown adipocyte gene in the absence of differentiation medium.

Also provided herein is an encapsulation system comprising a non-naturally occurring three-dimensional brown adipose derived stem cell aggregate. The three-dimensional brown adipose derived stem cell aggregate comprises brown adipose-derived stem cells that express one or more brown adipocyte gene in the absence of differentiation medium.

Also provided herein is a method of making a non-naturally occurring three-dimensional brown adipose derived stem cell aggregate. The method comprises: loading brown adipose derived stem cells grown in a two-dimensional (2D) culture into a non-adherent culture plate, and centrifuging the non-adherent culture plate to uniformly position the brown adipose-derived stem cells in the non-adherent culture plate, thereby forming three-dimensional brown adipose derived stem cell aggregates.

Also provided herein is a method of making a three-dimensional brown adipose tissue in an encapsulation system. The method comprises: forming non-naturally occurring three-dimensional brown adipose derived stem cell aggregates, loading the non-naturally occurring three-dimensional brown adipose derived stem cell aggregates into the encapsulation system, differentiating the non-naturally occurring three-dimensional brown adipose derived stem cell aggregates into brown adipose tissue in a first differentiation medium, and differentiating the non-naturally occurring three-dimensional brown adipose derived stem cell aggregates into brown adipose tissue in a second differentiation medium.

Also provided herein is a method of treating a patient with a disorder. The method comprises: forming non-naturally occurring three-dimensional brown adipose derived stem cell aggregates; loading the non-naturally occurring three-dimensional brown adipose derived stem cell aggregates into an encapsulation system; differentiating the non-naturally occurring three-dimensional brown adipose derived stem cell aggregates into brown adipose tissue in a first differentiation medium; differentiating the non-naturally occurring three-dimensional brown adipose derived stem cell aggregates into brown adipose tissue in a second differentiation medium; and delivering the brown adipose tissue to the patient with the disorder.

The foregoing summary is illustrative only and is not intended to be in any way limiting. In addition to the illustrative examples and features described herein, further aspects, examples, objects and features of the disclosure will become fully apparent from the drawings and the detailed description and the claims.

### BRIEF DESCRIPTION OF THE DRAWINGS

The patent or patent application contains at least one drawing executed in color. Copies of this patent or patent application publication with color drawing(s) will be provided by the Office upon request and payment of the necessary fee.

Various aspects of non-naturally occurring 3D brown adipose-derived stem cell (BADSC) aggregates, methods of making the 3D BADSC aggregates, and methods of using the 3D BADSC aggregates are disclosed and described in this specification and can be better understood by reference to the accompanying figures, in which:

FIG. 1 shows thirteen human brown adipose-derived mesenchymal stem cell (BADSC) populations (BF-1 to BF-13) isolated from subcutaneous supraclavicular and mediastinal adipose tissue biopsies and evaluated for their ability to differentiate into brown adipocytes. These BADSC populations were evaluated via bright field (top panels) and oil red O staining (ORO) (middle panels).

FIG. 2A shows UCP-1 expression for BADSC population BF-1 before differentiation in AD-1 culture medium (Pre Diff AD-1); BADSC population BF-1 post differentiation in AD-1 culture medium (Post Diff AD-1); BADSC population BF-1 before differentiation in AD-2 culture medium (Pre Diff AD-2); and BADSC population BF-1 after differentiation in AD-2 culture medium (Post Diff AD-2). Human brown adipose tissue was used as a positive control (Human BAT). Human white adipose tissue was used as a negative control (SubQ WAT and Visc. WAT).

FIG. 2B shows the expression of UCP1, FABP4, ADIPSIN, and LEPTIN mRNA via qPCR for the BADSC population BF-1, 15 days after differentiation in either StemPro™, AD-1, or AD-2 culture medium.

FIG. 2C shows the BADSC population BF-1 differentiated in AD-2 culture medium. Fifteen days after differentiation induction, cells were fixed and immunostained for Perilipin (green), UCP1 (red), and counterstained with DAPI (blue).

FIG. 2D shows the BADSC population BF-1 differentiated in AD-2 culture medium. Fifteen days after differentiation induction, cells were fixed and immunostained for Mitochondria (green), UCP1 (red), and counterstained with DAPI (blue).

FIG. 2E shows the quantification of adipocyte differentiation efficiency (% differentiation) for the BADSC population BF-1 defined as the percentage of perilipin positive cells using DAPI to quantify the total number of cells per field of view. FIG. 2E also shows the quantification of brown adipocyte differentiation efficiency (% Brown) for the BADSC population BF-1 defined as the percentage of perilipin positive cells that are positive for UCP1.

FIG. 3 shows gene expression levels of adipocyte markers (PPARa, PPARg, PGC1b, PRDM16, CEBPd, CEBPb, CEBPa, and TFAM) and brown adipocyte markers (PGC1a) determined by qPCR for (1) BADSC population BF-1 in 2D, (2) BADSC population BF-1 in 3D at 24 hours; and (3) BADSC population BF-1 in 3D at 48 hours.

FIG. 4A shows a schematic diagram of the three-step 3D brown adipocyte differentiation protocol in an encapsulation system.

FIG. 4B shows a micrograph (5×'magnification) of BAGs, 24 hours post formation.

FIG. 4C shows a micrograph (5× magnification) of BAGs, after collection.

FIG. 4D shows a photograph of the encapsulation medical device, Encaptra® EN20.

FIG. 4E shows a photograph of the encapsulation medical device, Encaptra® EN20, loaded with BAGs.

FIG. 4F shows a micrograph (10× magnification) of live BAGs differentiating inside the encapsulation medical device, Encaptra® EN20.

FIG. 4G shows cross sections of BAGs differentiating inside the encapsulation medical device, Encaptra® EN20. The cross sections were shown using (1) hematoxylin and eosin staining; (2) bright field; (3) immunostaining for Perilipin (green), UCP1 (red) and counterstaining with DAPI (blue); (4) staining with DAPI (blue); (5) immunostaining for Perilipin (green); and (6) immunostaining for UCP1 (red).

FIG. 4H shows gene expression levels of adipocyte markers (FABP4, ADIPSIN, PPARg, CEBPa, LEPTIN) and brown adipocyte markers (UCP1, PGC1a, ELOVL3, CIDEA, and COX10) determined by qPCR. RNA that was used for the qPCR was collected from BAGs at D0 (undifferentiated) and from the BAGs located within the encapsulation medical device, Encaptra® EN20, at D25 (see FIG. 4A).

FIG. 5A is a graph showing Glucose Tolerance Testing (GTT) of mice transplanted with BAT encapsulated in matrigel compared to mice transplanted with only matrigel.

FIGS. 5B-C are tables representing the data generated in the GTT experiment described in FIG. 5A.

### DETAILED DESCRIPTION

Certain exemplary aspects of the present disclosure will now be described to provide an overall understanding of the principles of the structure, function, manufacture, and use of the non-naturally occurring three-dimensional brown adipose-derived stem cell aggregates and methods disclosed herein. One or more examples of these aspects are illustrated in the accompanying drawings. Those of ordinary skill in the art will understand that the non-naturally occurring three-dimensional brown adipose-derived stem cell aggregates and methods specifically described herein and illustrated in the accompanying drawings are non-limiting exemplary aspects and that the scope of the various examples of the present disclosure is defined solely by the claims. The features illustrated or described in connection with one exemplary aspect may be combined with the features of other aspects. Such modifications and variations are intended to be included within the scope of the present disclosure.

### Non-Naturally Occurring Three-Dimensional Brown Adipose Derived Stem Cell Aggregates and Methods of Making the Non-Naturally Occurring 3D BAGS

Non-naturally occurring three-dimensional BADSC aggregates or “BAGs” are disclosed herein. The BAGs are 3D structures formed from BADSC after the BADSC are removed from their two-dimensional (2D) culture in cell adherent tissue culture flasks, added to non-adherent culture plates, and centrifuged. After centrifugation, the aggregates are uniform. Uniform cell aggregates provide a more efficient and consistent differentiation and are easier to load into encapsulation systems. Furthermore, uniform aggregation provides a more accurate cell number and a more accurate dosage.

BADSCs grown in 2D are the natural state of the BADSC whenever the cells expand in tissue adherent cell culture flasks. BADSCs cultured in growth media in 2D are multipotent and function as a stem cell. The BADSC grown in 2D cannot form aggregates because they attach to the cell culture flask and then differentiate into an unwanted non-adipose cell type and ultimately induce apoptotic cascade and cell death.

BADSC cannot form cell aggregates in 2D culture, but whenever the BADSC are removed from their 2D tissue adherent environment and placed in a non-adherent environment, the cells form 3D aggregates, as described above. The BADSC when aggregated form clusters of cells that are able to communicate with each other and their environment in 3D.

The BAGs can be expanded and further aggregated to become artificial brown adipose tissue or artificial white adipose tissue. The BAGs can become white adipose tissue if differtiated in AD-1. AD-1 is a serum based differentiation medium composed of DMEM low glucose (Gibco, Thermo Fisher Scientific) supplemented with 10% fetal bovine serum (FBS, HyClone, GE Healthcare, Life Sciences, Little Chalfont, Buckinghamshire, UK), 5 μM dexamethasone (MP Biomedicals, Santa Ana, California, USA), 500 μM 3-isobutyl-1-methylxanthine (IBMX, Sigma-Aldrich, St. Louis, Missouri, USA), 860 nM insulin (Gibco, Thermo Fisher Scientific), 125 nM indomethacin (Sigma-Aldrich), 1 nM triiodothyronine (T3, Sigma-Aldrich), 1 μM rosiglitazone (Sigma-Aldrich), 100 units/ml of penicillin, 100 μg/ml of streptomycin (Gibco, Thermo Fisher Scientific), and 2 mM L-glutamine (Gibco, Thermo Fisher Scientific)

The BAGs can become brown adipose tissue if differentiated in AD-2. AD-2 is a two-step xeno-free, serum free, chemically defined differentiation medium. In a first step, BADSC are grown in a first differentiation medium, AD-2 DIFF-1 culture medium, which comprises DMEM/Ham's F12 Media (1:1) (Lonza Group AG, Basel, Switzerland), 25 mM HEPES Buffer (Lonza Group AG), 2 mM L-glutamine (Gibco, Thermo Fisher Scientific), 1 μM dexamethasone (MP Biomedicals), 100 μM IBMX (Sigma-Aldrich), 860 nM insulin (Gibco, Thermo Fisher Scientific), 0.2 nM T3 (Sigma-Aldrich), 10 μg/ml apo-transferrin (Sigma-Aldrich), 100 units/ml of penicillin and 100 μg/ml of streptomycin (Gibco, Thermo Fisher Scientific). In a second step, after three days the AD-2 DIFF-1 culture medium was replaced with a second differentiation medium, AD-2 DIFF-2, a xeno-free, serum free, chemically defined differentiation medium, which comprises DMEM/Ham's F12 Media (1:1) (Lonza Group AG), 25 mM HEPES Buffer (Lonza Group AG), 2 mM L-glutamine (Gibco, Thermo Fisher Scientific), 860 nM insulin (Gibco, Thermo Fisher Scientific), 0.2 nM T3 (Sigma-Aldrich), 10 μg/ml apo-transferrin (Sigma-Aldrich), 100 units/ml of penicillin and 100 μg/ml of streptomycin (Gibco, Thermo Fisher Scientific) and 100 nM rosiglitazone.

These BAGs can serve as cell factories that can produce white or brown extracellular biologics (e.g., exosomes, microRNAs, cytokines, proteins, adipokines).

### Gene Expression of 3D BAGS

BAGs upregulate adipocyte markers (PPARα, PPARγ, PGC1β, PRDM16, CEBPd, CEBPb, CEBPa, and TFAM) and brown adipocyte markers (PGC1α) in the absence of differentiation medium.

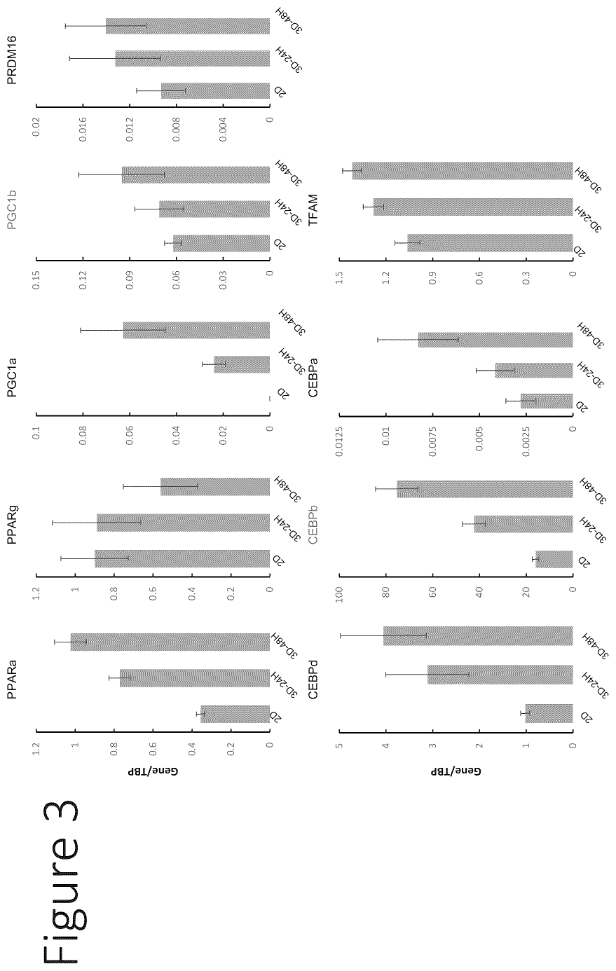

The formation of BAGs resulted in an increased expression of transcription factors and co-factors from the CEBP and PPAR families, which are master regulators of adipogenesis and browning (FIG. 3). “Browning” refers to the BAGs ability to express UCP-1 post-differentiation in AD-2 medium.

The early adipocyte differentiation transcription factors CEBPD and CEBPB were increased after 24 hours in 3D culture whereas CEBPA was significantly increased after 48 hours in 3D culture. PPARα, a master regulator of fatty acid oxidation, and PGC1α, a regulator of mitochondrial respiration and heat production in brown adipocyte, were both increased after 24 hours in 3D cultures whereas, no significant changes in the expression of PPARγ, PRDM16, TFAM or PGC1β were observed.

These data suggest that the formation of 3D BAGs commits BADSC aggregates to adipogenesis and a brown adipose phenotype and so the BAGs have started down the path of brown adipose differentiation in the absence of adipocyte differentiation medium.

### Encapsulation Systems as Delivery Systems for BAT

Transplanting brown adipose tissue (BAT) into humans, in order to increase BAT mass and/or activity, has emerged as a potential way to increase energy expenditure by energy wasting. This approach of transplanting BAT into humans can be used to treat metabolic disorders, endocrine disorders, cardiovascular disorders, and liver diseases. Therfore, a method of delivering BAT for transplantation using 3D BAGs loaded into encapsulation systems was sought and is disclosed herein.

Several different encapsulation systems can be loaded with BAGs and used to deliver the BAT for transplantation such as alginate microcapsules, cellulose hydrogels, red blood cells, porous polymer membranes, 3D biological scaffolds, Afibromer™ polymers (Sigilon Therapeutics, Cambridge, Massachussetts, USA), PEG-based hydrogels, non-hydrogel beads, and matrigel.

The encapsulation systems described herein allow the BAGs to produce extracellular factors that can interact with the host environment, such as proteins, cytokines, microRNAs, cytokines, exosomes, and cell specific secretome.

The encapsulation systems described herein are manufactured from implantable-grade materials or biologics and are selected for long-term biocompatibility.

The encapsulation systems described herein provide a bidirectional exchange of nutrients and molecules such as glucose, fatty acids, cytokines, adipokines, and hormones.

In certain examples, the encapsulation system can be an encapsulation medical device. In other examples, the encapsulation medical device can be an FDA-approved, immune-protecting, easily retrievable encapsulation medical device, such as the Encaptra® Drug Delivery System (Viacyte, San Diego, California, USA). This device is manufactured from implant-grade materials specifically selected for long-term biocompatibility and allows for bidirectional exchange of nutrients and molecules such as glucose, fatty acids, and hormones. The encapsulation medical device provides a barrier between the host and the transplanted cells and therefore should prevent immune rejection of BAT while increasing safety and preventing transplanted cells to migrate out of the encapsulation medical device.

### Method of Making 3D BAT in an Encapsulation System

Disclosed herein is a method of making 3D BAT in an encapsulation system. The method comprises (1) forming non-naturally occurring three-dimensional brown adipose derived stem cell aggregates, (2) loading the non-naturally occurring three-dimensional brown adipose derived stem cell aggregates into the encapsulation system, (3) differentiating the non-naturally occurring three-dimensional brown adipose derived stem cell aggregates into brown adipose tissue in a first differentiation medium, and (4) differentiating the non-naturally occurring three-dimensional brown adipose derived stem cell aggregates into brown adipose tissue in a second differentiation medium. The “first differentiation medium” can also be referred to herein as AD-2 DIFF-1 culture medium. The “second differentiation medium” can also be referred to herein as AD-2 DIFF-2 culture medium.

### Methods of Treatment

Methods of treating patients with disorders are disclosed herein. Methods of treating patients with metabolic disorders, endocrine disorders, cardiovascular disorders, and liver diseases are disclosed herein. Examples of metabolic disorders can include, but are not limited to, diabetes and obesity. Examples of endocrine disorders can include, but are not limited to, acromegaly, Addison's Disease, adrenal cancer, adrenal disorders, anaplastic thyroid cancer, Cushing's Syndrome, De Quervain's Thyroiditis, diabetes (e.g., type 1 diabetes, type 2 diabetes, gestational diabetes, maturity onset diabetes of the young), follicular thyroid cancer, goiters, Graves' Disease, growth disorders, growth hormone deficiency, Hashimoto's Thyroiditis, heart disease, Hurthle Cell Thyroid Cancer, hyperglycemia, hyperparathyroidism, hyperthyroidism, hypoglycemia, hypoparathyroidism, hypothyroidism, low testosterone, medullary thyroid cancer, MEN 1, MEN 2A, MEN 2B, menopause, metabolic syndrome, obesity, osteoporosis, papillary thyroid cancer, parathyroid diseases, pheochromocytoma, pituitary disorders, pituitary tumors, polycystic ovary syndrome, prediabetes, reproduction, silent thyroiditis, thyroid cancer, thyroid diseases, thyroid nodules, thyroiditis, turner syndrome, insulin resistance, hypertension, central obesity, hypertriglyceridemia (e.g., high serum triglycerides), dyslipidemia, low serum HDL, lipodystrophy. Examples of cardiovascular disorders can include, but are not limited to, coronary artery disease, peripheral artery disease, carotid artery disease, peripheral artery (arterial) disease, aneurysm, atherosclerosis, renal artery disease, Raynaud's disease (Raynaud's phenomenon), Buerger's disease, peripheral venous disease, cerebrovascular disease (e.g., stroke), venous blood clots, and blood clotting disorders, cardiomyopathy, hypertensive heart disease (e.g., diseases of the heart secondary to high blood pressure or hypertension). Examples of liver disease can include, but are not limited to, simple fatty liver disease, nonalcoholic steatohepatitis (NASH), and alcohol-related fatty liver disease (ALD).

Disclosed herein is a method of treating a patient with a metabolic disorder. The method comprises: forming non-naturally occurring three-dimensional brown adipose derived stem cell aggregates; loading the non-naturally occurring three-dimensional brown adipose derived stem cell aggregates into an encapsulation system; differentiating the non-naturally occurring three-dimensional brown adipose derived stem cell aggregates into brown adipose tissue in a first differentiation medium; differentiating the non-naturally occurring three-dimensional brown adipose derived stem cell aggregates into brown adipose tissue in a second differentiation medium; and delivering the brown adipose tissue to the patient with a metabolic disorder.

Disclosed herein is a method of treating a patient with obesity. The method comprises: forming non-naturally occurring three-dimensional brown adipose derived stem cell aggregates; loading the non-naturally occurring three-dimensional brown adipose derived stem cell aggregates into an encapsulation system; differentiating the non-naturally occurring three-dimensional brown adipose derived stem cell aggregates into brown adipose tissue in a first differentiation medium; differentiating the non-naturally occurring three-dimensional brown adipose derived stem cell aggregates into brown adipose tissue in a second differentiation medium; and delivering the brown adipose tissue to the patient with obesity.

Disclosed herein is a method of treating a patient with an endocrine disorder. The method comprises: forming non-naturally occurring three-dimensional brown adipose derived stem cell aggregates; loading the non-naturally occurring three-dimensional brown adipose derived stem cell aggregates into an encapsulation system; differentiating the non-naturally occurring three-dimensional brown adipose derived stem cell aggregates into brown adipose tissue in a first differentiation medium; differentiating the non-naturally occurring three-dimensional brown adipose derived stem cell aggregates into brown adipose tissue in a second differentiation medium; and delivering the brown adipose tissue to the patient with an endocrine disorder.

Disclosed herein is a method of treating a patient with a cardiovascular disorder. The method comprises: forming non-naturally occurring three-dimensional brown adipose derived stem cell aggregates; loading the non-naturally occurring three-dimensional brown adipose derived stem cell aggregates into an encapsulation system; differentiating the non-naturally occurring three-dimensional brown adipose derived stem cell aggregates into brown adipose tissue in a first differentiation medium; differentiating the non-naturally occurring three-dimensional brown adipose derived stem cell aggregates into brown adipose tissue in a second differentiation medium; and delivering the brown adipose tissue to the patient with a cardiovascular disorder.

Disclosed herein is a method of treating a patient with liver disease. The method comprises: forming non-naturally occurring three-dimensional brown adipose derived stem cell aggregates; loading the non-naturally occurring three-dimensional brown adipose derived stem cell aggregates into an encapsulation system; differentiating the non-naturally occurring three-dimensional brown adipose derived stem cell aggregates into brown adipose tissue in a first differentiation medium; differentiating the non-naturally occurring three-dimensional brown adipose derived stem cell aggregates into brown adipose tissue in a second differentiation medium; and delivering the brown adipose tissue to the patient with liver disease.

### Materials and Methods of the Invention

Various aspects of the invention according to the present disclosure include, but are not limited to, the aspects listed in the following numbered clauses:

1. A non-naturally occurring three-dimensional brown adipose derived stem cell aggregate wherein the three-dimensional brown adipose derived stem cell aggregate comprises brown adipose-derived stem cells that express one or more brown adipocyte gene in the absence of differentiation medium.

2. The non-naturally occurring three-dimensional brown adipose derived stem cell aggregate of clause 1, wherein the one or more brown adipocyte gene is selected from a group consisting of PPARα, PPARγ, PGC1β, PRDM16, CEBPD, CEBPB, CEBPA, TFAM, PGC1α, and PGC1β.

3. The non-naturally occurring three-dimensional brown adipose derived stem cell aggregate of any one of clauses 1-2, wherein the aggregate forms in a non-adherent environment.

4. The non-naturally occurring three-dimensional brown adipose derived stem cell aggregate of any one of clauses 1-3, wherein aggregate produces extracellular biologics selected from a group consisting of exosomes, microRNA, cytokines, proteins, and adipokines.

5. An encapsulation system comprising the non-naturally occurring three-dimensional brown adipose derived stem cell aggregate of any one of clauses 1-4.

6. The encapsulation system of clause 5, wherein the encapsulation system is selected from the group consisting of alginate microcapsules, cellulose hydrogels, red blood cells, porous polymer membranes, 3D biological scaffolds, polymers, PEG-based hydrogels, non-hydrogel beads, and matrigel.

7. The encapsulation system of clause 5, wherein the encapsulation system is an encapsulation medical device.

8. A method of making a non-naturally occurring three-dimensional brown adipose derived stem cell aggregate, the method comprising:
loading brown adipose derived stem cells grown in a two-dimensional (2D) culture into a non-adherent culture plate; and
centrifuging the non-adherent culture plate to uniformly position the brown adipose-derived stem cells in the non-adherent culture plate, thereby forming three-dimensional brown adipose derived stem cell aggregates.

9. The method of clause 8, further comprising:
prior to the loading, culturing the brown adipose derived stem cells in a two-dimensional (2D) culture using growth medium under normoxia or hypoxia.

10. A method of making a three-dimensional brown adipose tissue in an encapsulation system, the method comprising:
forming non-naturally occurring three-dimensional brown adipose derived stem cell aggregates;
loading the non-naturally occurring three-dimensional brown adipose derived stem cell aggregates into the encapsulation system;
differentiating the non-naturally occurring three-dimensional brown adipose derived stem cell aggregates into brown adipose tissue in a first differentiation medium; and
differentiating the non-naturally occurring three-dimensional brown adipose derived stem cell aggregates into brown adipose tissue in a second differentiation medium.

11. The method of clause 10, wherein the encapsulation system is selected from the group consisting of alginate microcapsules, cellulose hydrogels, red blood cells, porous polymer membranes, 3D biological scaffolds, polymers, PEG-based hydrogels, non-hydrogel beads, and matrigel.

12. The method of clause 10, wherein the encapsulation system is an encapsulation medical device.

13. The method of any one of clauses 10-12, wherein the first differentiation medium comprises dexamethasone, IBMX, and T3.

14. The method of any one of clauses 10-13, wherein the second differentiation medium comprises T3 and rosiglitazone.

15. A method of treating a patient with a disorder, the method comprising:
forming non-naturally occurring three-dimensional brown adipose derived stem cell aggregates;
loading the non-naturally occurring three-dimensional brown adipose derived stem cell aggregates into an encapsulation system;
differentiating the non-naturally occurring three-dimensional brown adipose derived stem cell aggregates into brown adipose tissue in a first differentiation medium;
differentiating the non-naturally occurring three-dimensional brown adipose derived stem cell aggregates into brown adipose tissue in a second differentiation medium; and
delivering the brown adipose tissue to the patient with the disorder.

16. The method of clause 15, wherein the encapsulation system is selected from the group consisting of alginate microcapsules, cellulose hydrogels, red blood cells, porous polymer membranes, 3D biological scaffolds, polymers, PEG-based hydrogels, non-hydrogel beads, and matrigel.

17. The method of clause 15, wherein the encapsulation system is an encapsulation medical device.

18. The method of any one of clauses 15-17, wherein the first differentiation medium comprises dexamethasone, IBMX, and T3.

19. The method of any one of clauses 15-18, wherein the second differentiation medium comprises T3 and rosiglitazone.

20. The method of any one of clauses 15-19, wherein the disorder is a metabolic disorder, an endocrine disorder, a cardiovascular disorder, or a liver disease.

21. The method of clause 20, wherein the metabolic disorder is obesity or diabetes.

### Definitions

In addition to the definitions previously set forth herein, the following definitions are relevant to the present disclosure:

The singular forms “a,” “an,” and “the” include plural references, unless the context clearly dictates otherwise.

A “2-dimensional (2D) culture” refers to cells spreading throughout the surface of a cell culture plate and adhering to the surface of the cell culture plate.

A “3-dimensional (3D) culture” refers to cells that do not adhere to the surface of a cell culture plate and instead associate with each other, thereby forming cellular aggregates.

Any numerical range recited in this specification describes all sub-ranges of the same numerical precision (i.e., having the same number of specified digits) subsumed within the recited range. For example, a recited range of “1.0 to 10.0” describes all sub-ranges between (and including) the recited minimum value of 1.0 and the recited maximum value of 10.0, such as, for example, “2.4 to 7.6,” even if the range of “2.4 to 7.6” is not expressly recited in the text of the specification. Accordingly, the Applicant reserves the right to amend this specification, including the claims, to expressly recite any sub-range of the same numerical precision subsumed within the ranges expressly recited in this specification. All such ranges are inherently described in this specification such that amending to expressly recite any such sub-ranges will comply with written description, sufficiency of description, and added matter requirements, including the requirements under 35 U.S.C. § 112(a) and Article 123(2) EPC. Also, unless expressly specified or otherwise required by context, all numerical parameters described in this specification (such as those expressing values, ranges, amounts, percentages, and the like) may be read as if prefaced by the word “about,” even if the word “about” does not expressly appear before a number. Additionally, numerical parameters described in this specification should be construed in light of the number of reported significant digits, numerical precision, and by applying ordinary rounding techniques. It is also understood that numerical parameters described in this specification will necessarily possess the inherent variability characteristic of the underlying measurement techniques used to determine the numerical value of the parameter.

Any patent, publication, or other disclosure material identified herein is incorporated by reference into this specification in its entirety unless otherwise indicated, but only to the extent that the incorporated material does not conflict with existing descriptions, definitions, statements, or other disclosure material expressly set forth in this specification. As such, and to the extent necessary, the express disclosure as set forth in this specification supersedes any conflicting material incorporated by reference. Any material, or portion thereof, that is said to be incorporated by reference into this specification, but which conflicts with existing definitions, statements, or other disclosure material set forth herein, is only incorporated to the extent that no conflict arises between that incorporated material and the existing disclosure material. Applicants reserve the right to amend this specification to expressly recite any subject matter, or portion thereof, incorporated by reference herein.

The details of one or more aspects of the present disclosure are set forth in the accompanying examples below. Although any materials and methods similar or equivalent to those described herein can be used in the practice or testing of the present disclosure, specific examples of the materials and methods contemplated are now described. Other features, objects and advantages of the present disclosure will be apparent from the description. In the description examples, the singular forms also include the plural unless the context clearly dictates otherwise. Unless defined otherwise, all technical and scientific terms used herein have the same meaning as commonly understood by one of ordinary skill in the art to which this present disclosure belongs. In the case of conflict, the present description will control.

### EXAMPLES

The present disclosure will be more fully understood by reference to the following examples, which provide illustrative, non-limiting aspects of the invention.

### Example 1—Differentiation of BADSC to Brown Adipocytes in Differentiation Medium Comprising Fetal Bovine Serum

BADSCs were isolated from fresh brown adipose tissue and were cultured for up to three passages. Cells were expanded in growth medium (GM) composed of Dulbecco's modified Eagle's medium (DMEM) low glucose (Gibco, Thermo Fisher Scientific, Waltham, Massachusetts, USA), supplemented with 10% human platelet lysate (Xcyte™ Plus Xeno-Free Supplement, iBiologics, Phoenix, Arizona, USA), 1% GlutaMAX™ Supplement (Gibco, Thermo Fisher Scientific), 1% Minimum Essential Medium Non-Essential Amino Acids (MEM-NEAA, Gibco, Thermo Fisher Scientific), 100 units/ml of penicillin and 100 μg/ml of streptomycin (Gibco, Thermo Fisher Scientific). Cells were seeded at a density of 3500 cells/cm² and medium was replaced every other day.

Adipocyte differentiation was induced two days after cells reached full confluency by addition of brown adipocyte differentiation medium 1 (AD-1). AD-1 is a serum based differentiation medium composed of DMEM low glucose (Gibco, Thermo Fisher Scientific) supplemented with 10% fetal bovine serum (FBS, HyClone, GE Healthcare, Life Sciences, Little Chalfont, Buckinghamshire, UK), 5 μM dexamethasone (MP Biomedicals, Santa Ana, California, USA), 500 μM 3-isobutyl-1-methylxanthine (IBMX, Sigma-Aldrich, St. Louis, Missouri, USA), 860 nM insulin (Gibco, Thermo Fisher Scientific), 125 nM indomethacin (Sigma-Aldrich), 1 nM triiodothyronine (T3, Sigma-Aldrich), 1 μM rosiglitazone (Sigma-Aldrich), 100 units/ml of penicillin, 100 μg/ml of streptomycin (Gibco, Thermo Fisher Scientific), and 2 mM L-glutamine (Gibco, Thermo Fisher Scientific).

### Example 2—Differentiation of BADSC to Brown Adipocytes in a 2-Step Serum-Free Chemically Defined Differentiation Medium

In order to develop a transplantable brown adipose tissue (BAT) for human applications, differentiation protocols applicable to cellular therapy in humans were sought.

BADSCs were isolated from fresh brown adipose tissue and were cultured for up to three passages. Cells were expanded in GM composed of DMEM low glucose (Gibco, Thermo Fisher Scientific), supplemented with 10% human platelet lysate (Xcyte™ Plus Xeno-Free Supplement, iBiologics), 1% GlutaMAX™ Supplement (Gibco, Thermo Fisher Scientific), 1% Minimum Essential Medium Non-Essential Amino Acids (MEM-NEAA, Gibco, Thermo Fisher Scientific), 100 units/ml of penicillin and 100 μg/ml of streptomycin (Gibco, Thermo Fisher Scientific). Cells were seeded at a density of 3500 cells/cm² and medium was replaced every other day.

Adipocyte differentiation was induced two days after cells reached full confluency by addition of brown adipocyte differentiation medium 2 (AD-2). AD-2 is a two-step xeno-free, serum free, chemically defined differentiation medium. In a first step, BADSC are grown in a first differentiation medium, AD-2 DIFF-1 culture medium, which comprises DMEM/Ham's F12 Media (1:1) (Lonza Group AG, Basel, Switzerland), 25 mM HEPES Buffer (Lonza Group AG), 2 mM L-glutamine (Gibco, Thermo Fisher Scientific), 1 μM dexamethasone (MP Biomedicals), 100 μM IBMX (Sigma-Aldrich), 860 nM insulin (Gibco, Thermo Fisher Scientific), 0.2 nM T3 (Sigma-Aldrich), 10 μg/ml apo-transferrin (Sigma-Aldrich), 100 units/ml of penicillin and 100 μg/ml of streptomycin (Gibco, Thermo Fisher Scientific). In a second step, after three days the AD-2 DIFF-1 culture medium was replaced with a second differentiation medium, AD-2 DIFF-2, a xeno-free, serum free, chemically defined differentiation medium, which comprises DMEM/Ham's F12 Media (1:1) (Lonza Group AG), 25 mM HEPES Buffer (Lonza Group AG), 2 mM L-glutamine (Gibco, Thermo Fisher Scientific), 860 nM insulin (Gibco, Thermo Fisher Scientific), 0.2 nM T3 (Sigma-Aldrich), 10 μg/ml apo-transferrin (Sigma-Aldrich), 100 units/ml of penicillin and 100 μg/ml of streptomycin (Gibco, Thermo Fisher Scientific) and 100 nM rosiglitazone.

In some examples, AD-2 can comprise human platelet lysate. In other examples, AD-2 does not comprise human platelet lysate.

The BADSC populations were differentiated in a xeno-free, serum free, chemically defined brown differentiation medium (AD-2 DIFF-1 and AD-2 DIFF-2) using a two-step method described above and its potency in generating brown adipocytes was compared to an FBS-based differentiation medium (AD-1) and to a commercially available adipogenic medium (StemPro™ Adipogenesis, Gibco, Thermo Fisher Scientific).

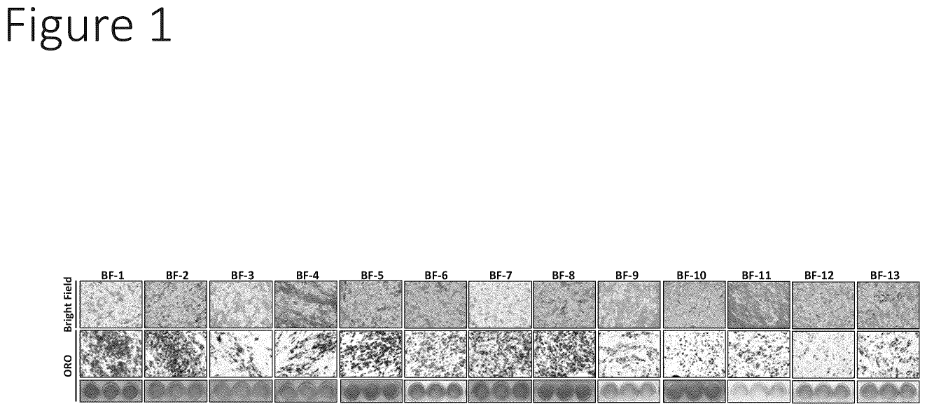

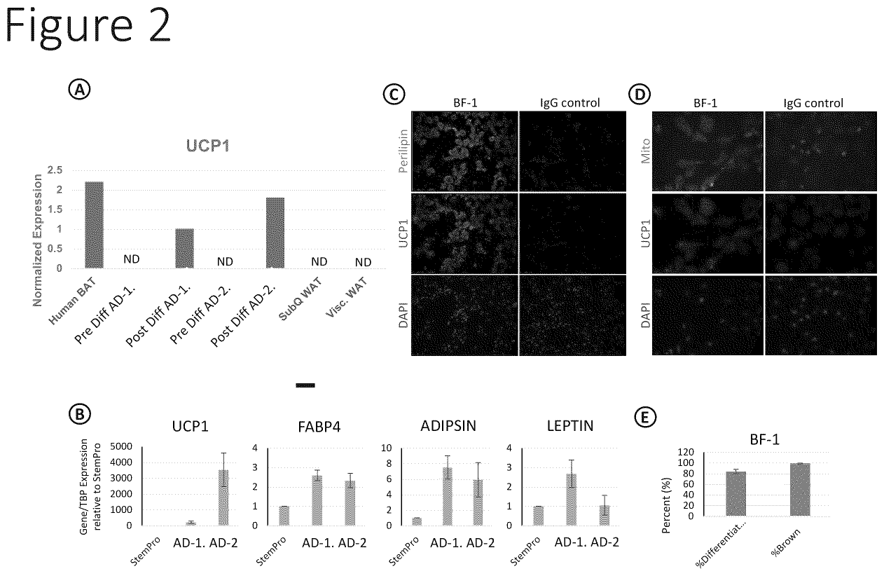

As demonstrated by the expression of the adipocyte markers FABP4 and adipsin (FIG. 2B), AD-1 and AD-2 adipogenic media were comparably efficient at converting BADSC into adipocytes and more efficient than the commercial adipogenic medium, StemPro™ (Gibco, Thermo Fisher Scientific) (FIG. 2B). Although AD-1 and AD-2 were equivalent in promoting adipocyte differentiation, adipocytes obtained in the xeno-free, serum free, chemically defined medium AD-2 were morphologically larger and contained larger lipid droplets (FIG. 1 shows cells cultured in AD-2; data not shown for cells cultured in AD-1). Differentiation using the AD-2 medium allowed for a much higher brown adipocyte differentiation than the AD-1 or the commercial adipogenic media.

Results also show that UCP1 gene expression was over 200 fold higher in AD-1 and over 3500 fold higher in AD-2 as compared to the commercial adipogenic medium, StemPro™ (FIG. 2B). Additionally, the expression of the white specific marker leptin was 1.5 fold lower in AD-2 than AD-1 confirming the superior efficiency of AD-2 to direct BADSC to a brown adipose phenotype (FIG. 2B).

Immunocytochemistry analysis of BADSC population BF-1 differentiated in AD-2 for 15 days showed that the adipocyte conversion rate i.e. the percentage of cell positive for the adipocyte marker perilipin, is very high with over 80% of the cells differentiating into adipocytes (FIGS. 2C and 2E). 98% of the differentiated cells (perilipin+cells) co-expressed the brown specific marker UCP1 (FIGS. 2C and 2E). This data confirmed the expression of UCP1 at the protein level (FIGS. 2C and 2D) and showed high yield of brown adipocyte conversion in the xeno-free, chemically defined differentiation medium. As expected, UCP1 protein localized in mitochondria as shown by the overlapping signals obtained when co-immunostaining differentiated BADSC for UCP1 and mitochondria (FIG. 2D).

The results in FIGS. 2A-E demonstrate that that the 2-step AD-2 differentiation medium (AD-2 DIFF-1 and AD-2 DIFF-2) promotes a stronger brown adipocyte differentiation compared to AD-1 differentiation medium and the commercial adipogenic media.

### Example 3—Method of Making 3D BAGs

Non-naturally occurring 3-dimensional BADSC aggregates or BAGs were formed in non-adherent culture plates, such as AggreWell™ 400Ex 6-well plates (StemCell Technologies, Vancouver, British, Columbia, Canada).

BADSCs were first cultured in 2D using growth media under normoxia or hypoxia until 80% confluency. The non-adherent plates were coated with a rinsing solution, such as AggreWell™ rinsing solution (StemCell Technologies) following manufacturer's instructions. After washing the non-adherent plates with GM, 12 ml of cell suspension containing 2.4 million cells/ml in GM was loaded to each well of the non-adherent plates. The non-adherent plates were then centrifuged at 500 g for 5 minutes using a swinging bucket centrifuge to allow the cells to settle uniformly into the microwells, resulting in a density of 1000 cells per microwell, thus creating uniform cellular aggregates. Without centrifugation, the non-naturally occurring 3-dimensional brown adipose-derived stem cell aggregates or BAGs will not be uniform.

The BAGs were then cultured in the a non-adherent culture plate, such as AggreWell™ 400Ex 6-well plates, at 37° C. in normoxia or hypoxia and 95% humidity for 24 hours in GM prior to harvest. Approximately 28200 BAGs were collected per non-adherent plate by gentle pipetting and resuspended in 800 μl of GM.

### Example 4—A Method of Making Three-Dimensional Brown Adipose Tissue in an Encapsulation System

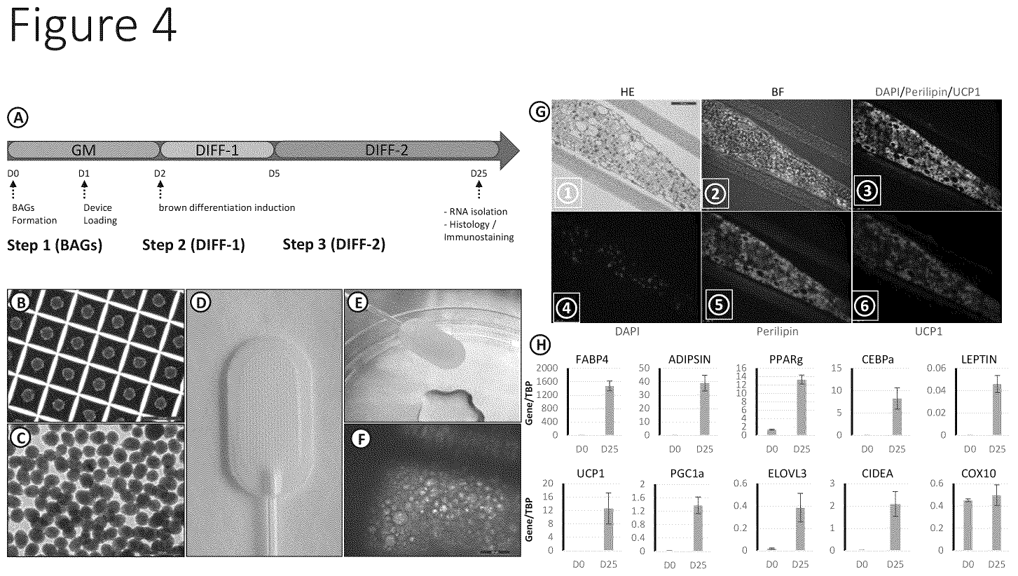

A differentiation protocol to efficiently differentiate BADSC into functional brown adipocytes in 3D culture within an encapsulation system, such as an encapsulation medical device, has been developed. This method, summarized in FIG. 4A, consists of 3 steps: (1) forming non-naturally occurring three-dimensional BADSC aggregates (BAGs) in growth medium (≈160 μm/aggregate) (FIG. 4B, 4C) and loading of BAGs into an encapsulation system, such as an encapsulation medical device (FIGS. 4D, 4E); (2) further differentiating the BAGS into brown adipose tissue (BAT) using the xeno-free, serum free, chemically defined AD-2-DIFF-1 medium; and (3) differentiating the BAGs into brown adipose tissue using the xeno-free, serum-free, chemically defined AD-2-DIFF-2 medium (FIG. 4F).

In step 1: BAGs were formed in AggreWell™ 400Ex 6-well plates (StemCell Technologies) using BADSC population BF-1. The optimal cell plating density, in order to generate uniform BAGs, was determined to be 1000 cells per microwell. BAGs were subsequently loaded into the encapsulation system, such as an encapsulation medical device. The BAGs suspension was loaded into an encapsulation device such as one Encaptra® EN20 (ViaCyte) encapsulation device, using a Sureflo® 20G catheter (Terumo Corporation, Tokyo, Japan). The device port was sealed with RTV Silicone Adhesive (NuSil Technology, Carpinteria, California, USA) and the encapsulated BAGs were cultured for 24 hours in 15 ml of GM in a 100 mm tissue culture dish. At that point, the BAGs merged and filled the entire volume of the encapsulation device. The resulting BAGs were highly uniform in size and shape, and uniform within and between experiments. Size can be easily modified by adjusting the cell seeding concentration formed in AggreWell™ 400Ex 6-well plates (StemCell Technologies). The optimal cell plating density in order to generate uniform BAGs was determined to be 1000 cells per microwell.

In step 2: the BAGs within the encapsulation medical device were differentiated for 3 days in vitro in a first differentiation medium called AD-2 DIFF-1 medium.

In step 3: the BAGs within the encapsulation medical device were further differentiated for 20 days in vitro in a second differentiation medium called AD-2 DIFF-2 medium.

Immunocytochemistry analysis showed that the non-naturally occurring brown adipose-derived stem cell aggregates comprising the BADSC population BF-1 efficiently differentiated into brown adipocytes in 3D inside the Encaptra® encapsulation medical device. BADSC BF-1 cells differenting in the encapsulation medical device formed a tissue-like structure visualized by hematoxylin and eosin staining highly enriched in brown adipocytes (UCP1 positive and Perilipin positive) containing high contents of mitochondria (FIG. 4G). These cells express high levels of adipocyte markers such as FABP4, adipsin, PPARg, CEBPa and leptin (FIG. 4H) and brown specific markers such as UCP1, PGC1a, CIDEA, ELOVL3, and COX10 (FIG. 4H) as compared to undifferentiated BAGs.

In conclusion, it was shown that non-naturally occurring BADSC aggregates represent a very promising source of transplantable brown adipose tissue to increase energy expenditure and to potentially treat metabolic disorders, endocrine disorders, cardiovascular disorders, and liver diseases. Moreover, the strategy to use encapsulation to deliver the non-naturally occurring BADSC aggregates represents a safe delivery system and will help accelerate the development of BAT therapies for human applications.

### Example 5—Evaluting Efficacy and Safety of BAGs Delivered in Matrigel

Male SCID-beige mice (C.B-Igh-1b/GbmsTac-Prkdcscid-LystbgN7), 8 weeks old (Taconic Biosciences) were singly housed at 25° C. and fed a high fat diet (HFT) comprising 60% fat (D12492, 60 kcal % fat [primarily lard], 20 kcal % carbohydrate). These mice have metabolic syndrome and cannot process glucose.

An encapsulation system was prepared by adding 1.6×10⁶ brown adipose derived stem cells (BADSCs) in 1 mL of 4 mg/mL matrigel (Corning® Matrigel® Matrix High Concentration (HC), Phenol-Red Free *LDEV-Free). The BADSCs were removed from their two-dimensional (2D) culture in cell adherent tissue culture flasks, added to non-adherent culture plates, and centrifuged. After centrifugation, the aggregates were uniform and added to the matrigel.

The encapsulation system (1 mL) was added to 20 wells of a 96-well plate (50 μL per well). After 1 hour of gelation, the encapsulation system became solid disks in the culture well. Growth media was added to the wells for 24 hours. The growth media was removed from the wells and AD-2 DIFF-1 was then added to the wells for 24 hours. The AD-2 DIFF-1 was removed from the wells and AD-2 DIFF-2 was then added to the wells for 14-21 days. After several days of culturing/differentiation, the encapsulation system comprising BAT forms a spherical shape (i.e. a bead). The beads contracted in size and reduced to 20-30 μl after the in vitro differentiation.

Forty beads were collected using a cell strainer. Forty beads comprise ˜3.2×10⁶ total cells that make up the encapsulated BAT. The beads were transferred into a 1.5 ml conical vial, and placed on ice. 100 μl of cold 10 mg/mL matrigel was added to the beads, mixed well, and kept on ice.

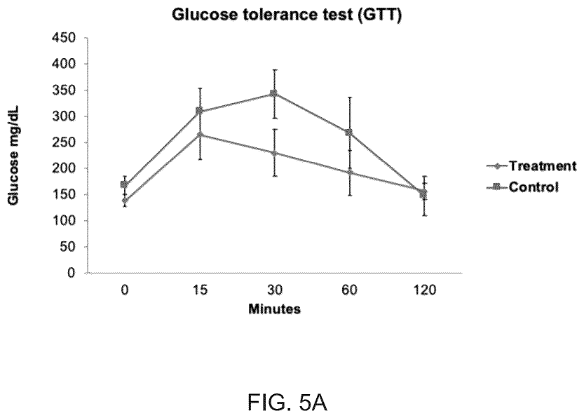

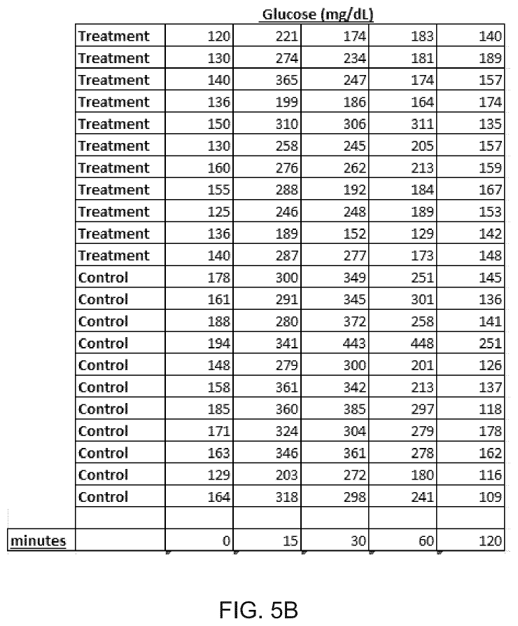

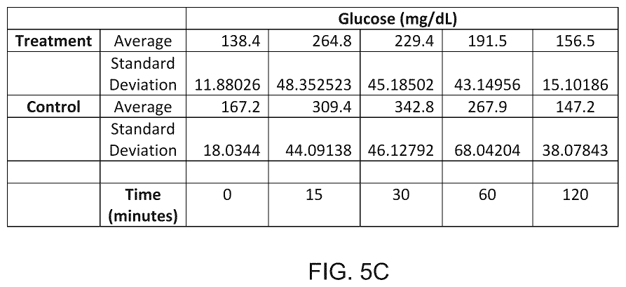

A small skin incision (˜5 mm) was made near in the Interscapular brown fat pads of twenty-two (22) SCID-beige mice. If additional space was needed, then dorsal subQ sites were used. A spatula was used to lift the skin off the underneath white fat layer. The 40 beads in matrigel were delivered to the incision site in 11 of the 22 mice (FIGS. 5A-C, Treatment group) using a modified 1 mL micropipette tip and the incision was sutured. Matrigel alone was delivered to the incision site in the other 11 mice (FIG. 5A-C, Control group) using a modified 1 mL micropipette tip and the incision was sutured.

Mice from the treatment group and mice from the control group were analyzed weekly to determine their ability to absorb glucose via a Glucose Tolerance Test (GTT). Prior to the analysis, these mice were fasted for 24 hours. After 24 hours, the mice were given an intraperitoneal (IP) injection of glucose (1 mg/g of body weight) and the amount of glucose that was absorbed was measured using a blood sample at 0, 15, 30, 60, and 120 minutes post glucose injection.

FIGS. 5A-C show that mice transplanted with BAT (treatment group) were better able to absorb glucose over the course of 60 minutes at 4-weeks post-treatment (8-weeks post time induction of obesity) as compared to mice that were not transplanted with BAT (control group).

### Example 6—Evaluting Efficacy and Safety of BAGs Delivered in Matrigel

Mice included in the GTT experiment described in Example 5 will also have their body weight monitored. To measure body weights, mice will be weighed weekly for a period of 3 months. Each week mice will be placed on a zeroed balance and their weights will be recorded. Mice transplanted with BAT (treatment group) will demonstrate a lower total body weight or will demonstrate a lower total weight gain compared to mice that were not transplanted with BAT (control group)

### Note Regarding Illustrative Examples

While the present disclosure provides descriptions of various specific aspects for the purpose of illustrating various examples of the present disclosure and/or its potential applications, it is understood that variations and modifications will occur to those skilled in the art. Accordingly, the invention or inventions described herein should be understood to be at least as broad as they are claimed, and not as more narrowly defined by particular illustrative examples provided herein.

1-21. (canceled)

22. A method of making a non-naturally occurring three-dimensional brown adipose derived stem cell aggregate consisting of brown adipose derived stem cells that express one or more brown adipocyte gene in the absence of differentiation medium, the method comprising:
loading brown adipose derived stem cells grown in a two-dimensional (2D) culture into a non-adherent culture plate; and
centrifuging the non-adherent culture plate to uniformly position the brown adipose derived stem cells in the non-adherent culture plate, thereby forming non-naturally occurring three-dimensional brown adipose derived stem cell aggregates, wherein the aggregates consist of brown adipose derived stem cells.

23. The method of claim 22, further comprising:
prior to the loading, culturing the brown adipose derived stem cells in a two-dimensional (2D) culture using growth medium under normoxia or hypoxia.

24. The method of claim 22, further comprising:
culturing the non-naturally occurring three-dimensional brown adipose derived stem cell aggregates in a non-adherent culture plate, such as an AggreWell™ 400Ex 6-well plate, at 37° C. in normoxia or hypoxia and 95% humidity for 24 hours in a growth medium after centrifuging and prior to harvesting the non-naturally occurring three-dimensional brown adipose derived stem cell aggregates.

25. The method of claim 22, further comprising:
loading the non-naturally occurring three-dimensional brown adipose derived stem cell aggregates into an encapsulation system;
differentiating the non-naturally occurring three-dimensional brown adipose derived stem cell aggregates into brown adipose tissue in a first differentiation medium; and
further differentiating the non-naturally occurring three-dimensional brown adipose derived stem cell aggregates into brown adipose tissue in a second differentiation medium.

26. The method of claim 25, wherein the encapsulation system is selected from the group consisting of alginate microcapsules, cellulose hydrogels, red blood cells, porous polymer membranes, 3D biological scaffolds, polymers, PEG-based hydrogels, non-hydrogel beads, and matrigel.

27. The method of claim 25, comprising
forming the non-naturally occurring three-dimensional brown adipose derived stem cell aggregates in a growth medium at ≈160 pm/aggregate before the loading of the non-naturally occurring three-dimensional brown adipose derived stem cell aggregates into the encapsulation system;
in the first differentiating step, using a xeno-free, serum free, chemically defined AD-2-DIFF-1 medium as the first differentiation medium; and
in the second differentiating step, using a xeno-free, serum-free, chemically defined AD-2-DIFF-2 medium as the second differentiation medium.

28. The method of claim 27, comprising
in the first differentiating step, differentiating the aggregates within the encapsulation system for 3 days in-vitro in the first differentiation medium; and
in the second differentiating step, further differentiating the aggregates within the encapsulation medical device for 20 days in-vitro in the second differentiation medium.

29. The method of claim 25,
wherein the encapsulation system is an encapsulation medical device; or
wherein the first differentiation medium comprises dexamethasone, 3-isobutyl-1-methylxanthine (IBMX), and triiodothyronine (T3); or
wherein the second differentiation medium comprises T3 and rosiglitazone.
